(19) United States
(12) Patent Application Publication
(10) Pub. No.: US 2006/0149196 A1
(43) Pub. Date: Jul. 6, 2006

(54) Incontinence protection device for men

(76) Inventors: Sten Bjornberg, Spanga (SE); Lars Mattason, Haljingajo (SE)

Correspondence Address: FASTH LAW OFFICES (ROLF FASTH), 26 PINECREST PLAZA, SUITE 2, SOUTHERN PINES, NC, 28387-4301, US

(21) Appl. No.: 10/543,945

(22) PCT Filed: Feb. 10, 2004
(86) PCT No.: PCT/SE04/00172

§ 371 Date: Aug. 1, 2005

(30) Foreign Application Priority Data
Feb. 11, 2003 (SE) 0300362-1

(51) Int. Cl. A61F 5/44

(52) U.S. Cl. 604/349

(57) ABSTRACT

Incontinence device for men formed as a pouch with an opening for insertion of penis or penis and scrotum. At one side of the pouch is one ore more elastic members located. The pouch is opened by pressing at the side edges of the elastic member so the pouch opens for insertion of penis or penis and scrotum. By the elastic restoration force of the elastic member the pouch is closed around penis or penis and scrotum and holds it in place. The elastic member is prepared so it always only bends in one direction when pressed upon from booth sides in its longitudinal direction which opens the pouch. The device is also equipped with friction layers on the inside of the pouch in order to further prevent penis from slipping out of the pouch.

Urine incontinence is social handicap and is increasing since it mainly affects elderly people and the ageing population is growing in the world.

To day 5% of the adult population is estimated to have some form of incontinence problems, even if the main part of the problem strikes women, it is also a big problem especially for elderly men.

Most incontinence protection devices on the market are developed and designed for women. These products are to a very large extend also used by men even though they in most cases are unsuitable for men's anatomy. The main difficulty is to keep the incontinence product in place especially when they are filled with urine, but also when they still are dry.

Men's anatomy often changes when they are getting older and it is rather common that elderly men have a retarded penis, this calls for a big flexibility in the design of male incontinence products in order to fit men's very different anatomy.

The biggest problem for incontinence products, especially made for men, are the difficulty to keep them in place. Movement and penis erection are some of the reasons for this problem. When the incontinence product are filled with urine the risk for penis to slip away from the device are even bigger.

This invention describes an incontinence protection device especially designed for men with very different anatomy, the device stays in place under difficult conditions and is because of its unique design comfortable to wear.

### PRIOR ART

Different types of incontinence protection devices for men are described in ea. U.S. Pat. No. 886,509 and 4.963,137, WO 8103609 and EP 0228353 these products are mainly made as pouches with an opening for penis or for penis and scrotum. All these devices have the same problem, the penis can easily slip out, especially when they are saturated with urine.

EP 0903131A describes another design were the device is foreseen with a pair of parallel elastic members located close to the opening of the pouch opposite to each other. These elastic members are designed to squeeze on penis by its elastic retention force and therefore keep the device in place, but it is not suited to fit around penis and scrotum which is a necessity for men with retarded penis.

If this design with a pair of elastic members are to be fitted around penis and scrotum the lower elastic member will press uncomfortably on scrotum, which is an extremely sensitive part of the body, and can cause skin irritation and scrape wounds on the wearers skin.

There is also a substantial risk that the lower elastic member gets in contact with scrotum and the testicles even when placed only over penis, especially if the penis is rather short and the location of the pouch therefore must be very close to scrotum and the testicles.

EP 0997126 A” describes a similar a similar solution as EP 090313 1A with a pair of elastic members located at the side portion of the incontinence pouch opposite of each other, this design has very similar problems as the design as EP 090313 1A and is not very well suited for locations around booth penis and scrotum.

This invention describes a solution to the above described problems by the use of elastic member(s) located only at one side of the pouch preferably, the front side (upside)of the pouch. Since the elastic member(s) are used to press only at the upside of penis the back side of the pouch is forming gently around scrotum, when the pouch is applied around booth penis and scrotum. This avoids unnecessary irritation and inconvenience on this very sensitive part of the body and gives a very good holding force on the pouch.

The use of elastic member on the front side of the pouch is also very well suited to use in applications on penis only, since the elastic member are located on the upside of penis, the backside of the pouch can form gently around a larger section of the underside of penis and cause no irritation to scrotum and the testicles.

The use of elastic member at only one side of the pouch will also lower the cost of the incontinence device and is easier to manufacture.

In some rare cases of men with extremely big belly, which partly can hang over penis, it can be an advantage to place the side of the pouch with the elastic member on the under side of pens in order to avoid contact with the elastic member to the belly.

In the description the vertical extension of the incontinence protection device is called “the length” and the horizontal extension of the device is called “the width”.

The invention is sometimes called, incontinence device, incontinence protection device, incontinence pouch or only pouch but it all means the same in this description.

With the expression absorption “bat” means the entire absorption core which can consist of one of several different absorbent layers. The expression elastic member can also be plural.

### DESCRIPTION OF FIGURES

FIG. 1 shows the incontinence protection device in an open position achieved by pushing at the elastic member 9 along the arrows A-A, 1 is the front side of the pouch and is sealed together with the backside 2 of the pouch along the side edges 3 and 4. 5 6 and 7 are friction layers located at the inside of the pouch, 8 shows that the backside of the pouch extends a bit over the front side in order to make the opening of the pouch easier.

FIG. 2 shows a schematic cut trough the incontinence device placed over penis 10, 9 is the elastic member which presses the incontinence device against penis, 5, 6 and 7 are friction layers which helps the incontinence device to stay in place.

FIG. 3 shows a schematic cut trough the incontinence device in an application which surrounds booth penis and scrotum were 12 in the elastic member, 11 friction layers, 13 a retarded penis and 14 scrotum.

FIG. 4 shows a schematic cut along the line B-B in FIG. 2 were 9 is the elastic member, 10 penis, 6a friction layer, 3 and 4 shows the sealing of the incontinence device along its side edges.

FIG. 5 shows a detailed cut through the incontinence device, 14 and 21 are liquid impermeable outer layers, 15 and 16 glue which seals the elastic member 9 with the outer layer 14 and the absorbent bat 17, 18 is another glue layer which seals the liquid permeable inner layer 19 together with the absorption bat 17, 21 is a liquid impermeable outer layer 25 a liquid permeable inner layer, 22 and 24 are layers of glue which binds the absorption bat 23 to with them, 5,6 and 7 are friction layers glued or sealed to the inner layers 19 and 25, all outer and inner layers are sealed together at the end portions 20 and 26 which is the side edges of the incontinence device.

FIG. 6 shows an example of a pre formed elastic member which only bends in one direction when pushed upon the side edges 27 and 29, 28 is the distance of the pre forming.

FIG. 7 shows an elastic member with cuts 31, which forces the elastic member to bend only in one direction when pushed at the side edges 30 and 32.

### DESCRIPTION OF THE INVENTION

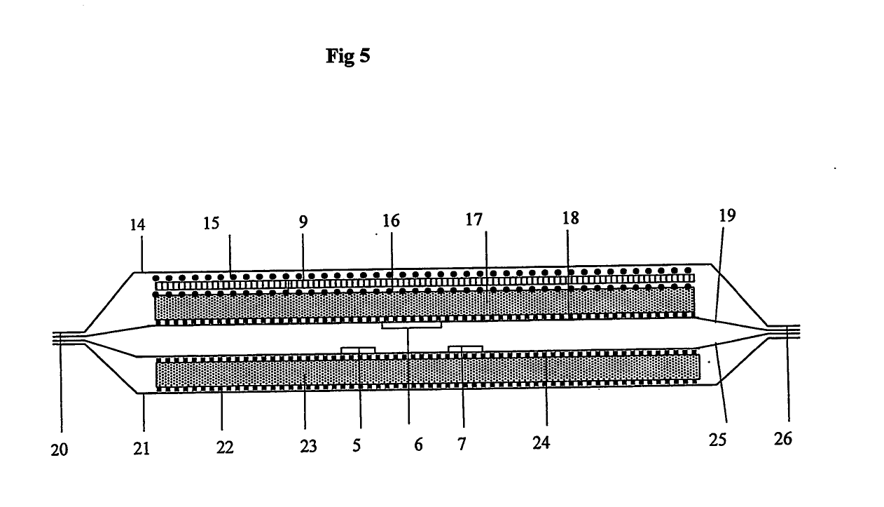

The incontinence device is formed as a pouch with an opening for insertion of penis or penis and scrotum. It can be manufactured in many ways, FIG. 1 shows one embodiment were the pouch is folded over its bottom section and sealed along its side edges 3 and 4, other types of folding are quite possible in order to form a pouch and are within the scoop of this invention. The material composition of the pouch are mainly made up as follows, fluid impermeable outer layers, as shown in FIG. 5, 14 and 2, these outer layers can me made of thin plastic film ea. polyethylene or copolymers thereof or other for the for the function suitable polymers, ea. breathable films with a water vapour permeability for example between 500-8,000 g H₂O/24 h /38° C. preferably between 400-5000 H₂O/24 h /38° C.

These types of plastic films are well known for the men skilled in the art and are used to a large extend in incontinence products, baby diapers and sanitary napkins. The thickness of these films can be between 0.005 to 0,09 mm preferably between 0,015-0,035 mm.

Foamed sheets with closed cells can also be used the thickness can then be anything from 0,035 mm to 2,5 mm preferably 0,15-1,5 mm.

At the outside of the water impermeable film a layer of nonwoven or tissue or soft paper can be laminated in order to get the incontinence device a less plastic look and be more comfortable against the skin of the wearer.

The inner layer of the pouch as shown in FIGS. 5, 19 and 25 are mainly made of a fluid permeable nonwoven this nonwoven can be made of many different fibres, ea. Polypropene, Polyethylene,, Polyester, Viscose, cotton rayon or mixes there of or other for the function suitable materials. The inner layer can also be made of perforated plastic film. The surface weight for these inner layers can be between 5-60 g/m2 or more, preferably 15-40 g/m2. These types of inner layers are well known for the men skilled in the art and are used to a large extend in incontinence products, baby diapers and sanitary napkins.

Between the outer and inner layer is an absorption bat located, FIGS. 5, 17 and 23. This absorption bat can be made of one or more layers of cellulose fluff, air laid cellulose, or combinations thereof, the surface weight of these layers can be anything between 30-2,000 g/m2 preferably 30-800 g/m2. The absorption bat can also contain Superabsorbents in a weight % of between 1-75% preferably 5-50%. The absorption bat can also be made op of several layers of soft paper or tissue, between these layers Superabsorbents can be encapsulated. These type of absorbent materials are well known by the men skilled in the art and are used to a large extend in incontinence products and sanitary napkins. Between the inner layer and the absorption bat can over the hole surface or partly an acquisition layer be located. These acquisition layers can be made of ea. Polyester, Polypropene fibres these types of acquisition layers are well known for the men skilled in the art and are used to a large extend in incontinence products and baby diapers.

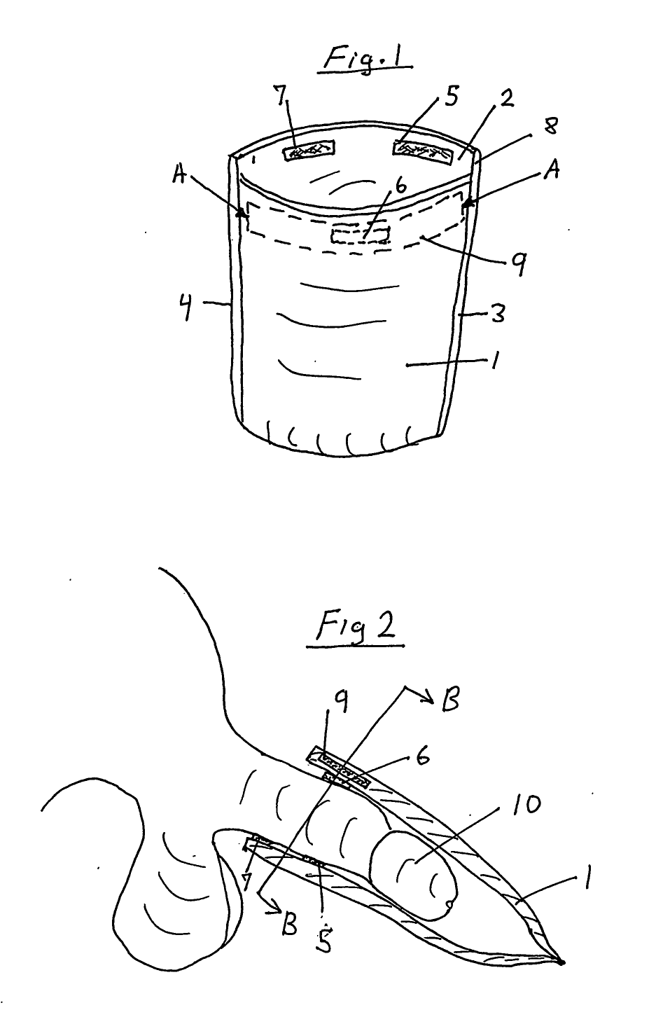

On the outside of the liquid permeable inner layer a friction layer can be located, FIGS. 1 and 2, 5, 6 and 7, these friction layers can be situated at one of the halves of the pouch or on booth sides, they can be situated directly against each other or at the side of each other and are preferably located in the vicinity of elastic member 9 but can also be located over a larger area of the inside of the pouch on either side or booth sides. The friction layers can extend in the longitudinal or transverse directions and can be partial or in a screen and be located so that the friction layer on one half of the incontinence layer has another extension than on the other half of the pouch in order for the friction layers to not get in contact with each other when the pouch is in a flat stage in order to avoid the friction layers to stick to each other if they are made tacky. These friction layers can be made of ea. expanded polyurethane, polyethylene, polypropylene, polystyrene or other for its purpose suitable materials characterised by being soft and flexible and having a high coefficient of friction against human skin. Other types of non sticking friction layers can also be suited ea. hot melt with friction increasing additives of ea. silica, chalk, or cold types of glue.

These types of friction additives are well known by the men skilled in the art. Examples of suitable friction member is Paltomal 17,190 from, Svenska lim, or MSA 36 or 38 from 3 M. These friction layers can be applied as one or more strings as a whole or as a screen.

All layers at the incontinence device are as a whole or partly sealed together with glue or heat sealing or combinations hereof The fluid impermeable outer layers 14 and 21 and the fluid permeable inner layers 19 and 25 in FIG. 5 are preferably extending outside of the absorption bats 17 and 23 and sealed together at their ends 20 and 26.

On one side preferably, the upper part of the front side of the pouch, is an elastic member 9 located, FIGS. 1, 2 and 5. The elastic member can also be made of more than one member, basically parallel to each other, but they can also be formed as to a net of elastic members extending over the whole or partly on one side of the pouch. The elastic member can be located between the outer layer 14 and the absorption bat 17 in FIG. 5 or between the inner layer 19 and the absorption bat, or between one or more of the absorption layers in the absorption bat if the bat is made up of two or more layers. The elastic member can also be located on the outside of the fluid impermeable outer layer 14 or at the outside of the fluid permeable inner layerl9, the elastic member will in the last case be in direct contact with the users penis. The elastic member is preferably located close to the opening in the pouch in the vicinity of the upper edge of the pouch but can also be located within a section extending from the upper edge on the pouch to ½ of the length of the pouch or preferably to ⅓ of the length of the pouch all calculated from the uppermost edge of the pouch. The elastic member can be partly or as a whole glued to the layers in the pouch and have an extension partly or over the whole width or at least ¼ of the total width at the position were the elastic device is located, the elastic device, is if it is shorter than the width of the pouch were the elastic member is located, is in this case preferably placed in the centre of the pouch in its transverse direction. The elastic member can be made of plastic or other elastic materials ea. rubber or wood or even metal, but preferably of plastic, the elastic member can be flat or half round with one flat and one rounded side or rounded on booth sides, but preferably flat shaped with a width between 3-50 mm preferably 5-25 mm and a thickness between 0.5-5 mm preferably 0.75-3 mm and be equipped with spoors or cuts FIG. 7, 31 in the length or transverse directions.

Many other shapes of the elastic member are possible within the scope of this invention, it can ea. be round, oval, square, triangular, tube formed or combinations thereof.

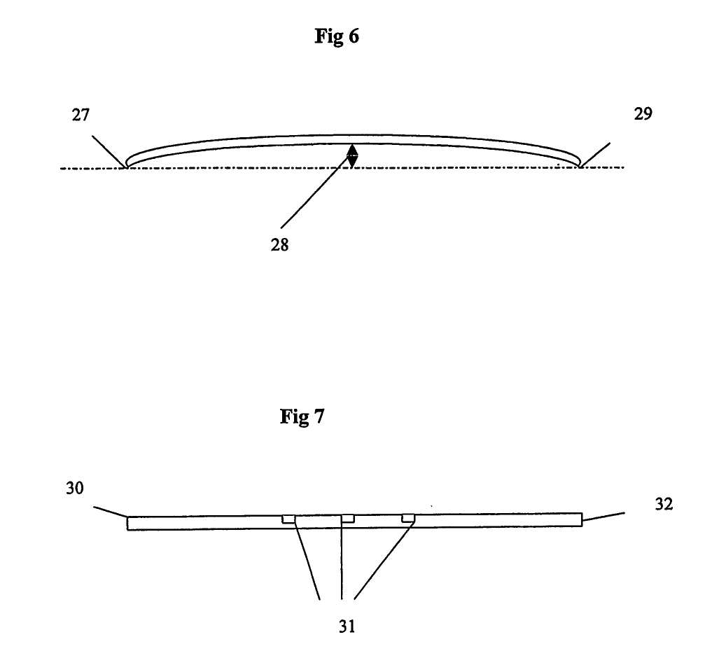

The elastic member can be flat, but can also be pre bent in its longitudinal direction, for example. when pressed upon its edges 27 and 29 in FIG. 6 it will bend in only one direction , the size of the pre bending shown in FIG. 6, 28 can be anything from 0.2 mm to 25 mm but preferably between 0.5- 7 mm, the pre bending is directed so when the pouch is opened the elastic member will bend in the direction to open the pouch so penis or penis and scrotum can be inserted in to the device, FIG. 1.

The elastic member can also be equipped with one or more transverse directed tracks or cuts FIG. 7, 31 the bending of the elastic member will then perform in one direction only, another practical way to get a pre bent elastic member is to roll up the elastic member directly after the extrusion as a roll, after cooling the molecule structure will have a memory and stay curved after the elastic member has been cut to its right length as shown in FIG. 6.

Another way to achieve a curved form is by cooling the extrusion tool, when the elastic member is extruded, with different temperatures on each side or the elastic member, a built in tension will then be achieved giving the elastic member a bent form. If the elastic member is injection moulded in can be made to any desired shape.

The end section at one side of the pouch can extend over the other side of the pouch, ea. FIG. 1, 8, in order to make it easier to open the pouch or to get longer contact sections on penis or scrotum.

Application and Handling of the Incontinence Device.

The application of the incontinence device can easily be made with only one hand in the following way.

Grab over the end parts of the elastic member with the thumb or middle fmger and press, the elastic member will then buckle and open the pouch, if needed the opposite half of the pouch can easily be opened with the fore finger with a slight push on the overlapping section of the pouch FIG. 1,8 at the same time.

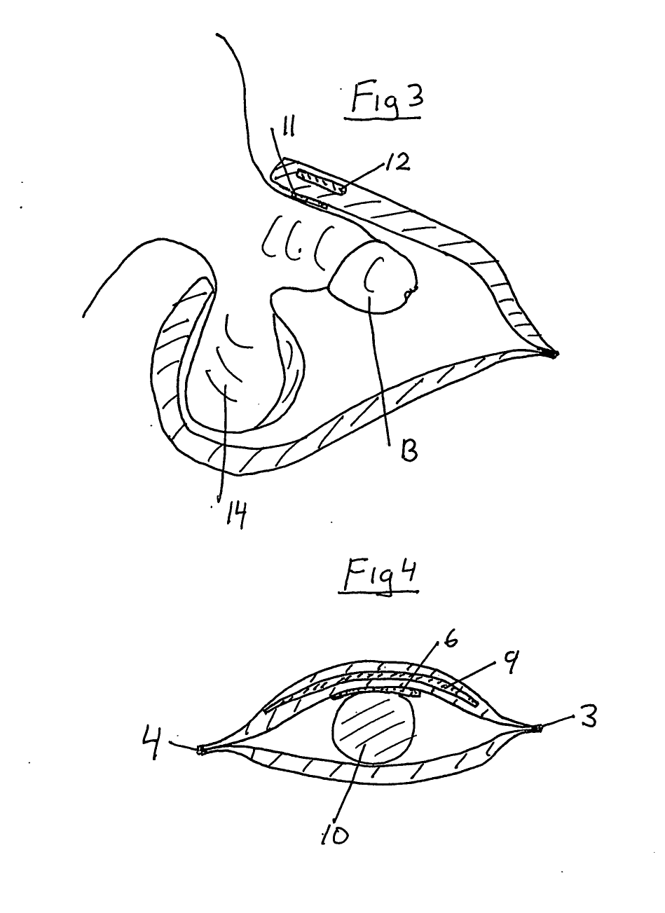

Place the pouch over penis and scrotum FIG. 3 or only penis FIG. 1 and release the grip on the elastic member. The opening of the pouch will then close around penis or penis and scrotum because of the elastic restoration force of the elastic member and effectively keep the incontinence device in place. The incontinence device can also easily be opened with two hands and placed over penis or penis and scrotum.

Example of a Product

The incontinence device is made as a pouch with a rhomboid design, FIG. 1, it has been sealed along its side edges 3 and 4 and folded over its bottom section, the length of the front section 1 is 100 mm and the length of the back section is 110 mm which means the overlapping section 8 is 10 mm. The width of the pouch in a flat stage is 85 mm at the top of the pouch and 75 mm at the bottom. The outer layer

FIG. 5 14 and 21 is made of 0.02 mm thick polyethylene film and the inner layer 19 and 25 are made of a polypropylene/polyethylene nonwoven with a surface weight of 20 g/m2. Between these two layers is an absorbent layer placed, 23 and 17, this layer is made of air laid cellulose with a surface weight of 350 g/m2.

Between the outer layer 14 and the absorbent layer 17 is an elastic plate placed, the plate is made of ABS plastic and have a width of 10 mm and a thickness of 1.5 mm and have the same length as the inside of the pouch were it is located, the elastic plate is placed 5 mm from the upper edge of the front section of the pouch.

The elastic plate is fastened on booth sides with hot melt glue. All layers are sealed together with hot melt glue, apart from the side edges 3 and 4 in FIG.1, which are heat sealed and consists of the outer layers 14 and 21 and inner layers 19 and 25 FIG. 5 which extends, 20 and 26, 5 mm outside of the absorption layers 23 and 17.

At the inside of the pouch on booth sides are friction layers 5, 6 and 7 FIG. 1 located. The friction layer 6 is 20 mm long and Smm wide and are placed over the elastic plate 9 in the centre of the pouch. The friction layers 5 and 7 are placed at a distance of 25 mm from each other and have booth a length of 15 mm and a width of 5 mm. They are placed in such a way so the friction layers opposite of each other does not come in contact with each other when the pouch is in a flat closed stage. These friction layers are made from a self tacking hot melt of type MSA 36 from 3 M.

1. Incontinence device for men formed as a pouch with an opening for insertion of penis or penis and scrotum. The incontinence device is mainly made of at least one outside layer of a liquid impermeable material and at least one inside layer of a liquid permeable material. Between these layers is an absorbent bat located characterised in that the pouch is equipped with one or more elastic members 9 located at one side of the pouch.

2. Incontinence device for men according to claim 1 and characterised in that the elastic member is pre bent 6.

3. Incontinence device for men according to claim 1-2 characterised in that the elastic member has a built in tension that makes it bend in only one direction when pressed upon 6.

4. Incontinence device for men according to claim 1-3 characterised in that the elastic member is flat round, oval, square, triangular, tube formed and combinations thereof.

5. Incontinence device for men according to claim 1-4 characterised in that the elastic member has one or more transverse cuts 31 on its backside.

6. Incontinence device for men according to claim 1-5 characterised in that the elastic member is located on the front side 9 of the pouch.

7. Incontinence device for men according to claim 1-6 characterised in that the pouch has one or more friction layers located at the inside of the pouch.

8. Incontinence device for men according to claim 1-7 characterised in that the end section at one side of the pouch can extend over the other side of the pouch 8.
